(19) United States
(12) Patent Application Publication
(10) Pub. No.: US 2013/0262259 A1
(43) Pub. Date: Oct. 3, 2013

(54) Method and System for Making Up Spectacles and Eyesight Testing through Public Network

(71) Applicants: Yiling Xie, Wenzhou (CN); Ji Jack Xie, Arcadia, CA (US)

(72) Inventors: Yiling Xie, Wenzhou (CN); Ji Jack Xie, Arcadia, CA (US)

(21) Appl. No.: 13/437,762

(22) Filed: Apr. 2, 2012

(51) Int. Cl. G06Q 30/06; G02C 13/00; A61B 3/11

(52) U.S. Cl. 705/26.5; 351/204; 351/178; 351/246

(57) ABSTRACT

A method of making a shelter spectacle for existing spectacles through a public network, includes the steps of gathering dimensional information on the existing spectacles worn by a user; sending the dimensional information on the existing spectacles to a fulfillment center through the public network; according to the dimensional information sent by the user, producing a shelter spectacles which fit the existing spectacles worn by the user; and sending the shelter spectacles back to the user.

### BACKGROUND OF THE PRESENT INVENTION

1. Field of Invention

The present invention relates to spectacles and more particularly to a method and a system for making up spectacles through a predetermined public network, wherein a user is to able to purchase shelter spectacles through the public network.

2. Description of Related Arts

Shelter spectacles have widely been utilized for a wide variety of purposes. For example, shelter spectacles may be purchased by users who are wearing existing spectacles for shielding sunlight. Furthermore, shelter spectacles may be manufactured to provide three-dimensional effect or to block radiation coming out from a monitor.

Conventionally, when a consumer wishes to purchase shelter spectacles, he or she must bring the existing spectacles to a spectacles store and select those shelter spectacles which fit the existing spectacles worn by the user. When there is no suitable shelter spectacles, store staff have to manually measure the dimension of the existing spectacles and record all the necessary dimensions. After that, the dimensions will be sent to the technicians for tailor-making shelter spectacles for the relevant user. This practice is rather time-consuming and highly inconvenient, particularly when the spectacles store is far away from where the user lives. Thus, the consumer must physically go to the spectacles store to see if the shelter spectacles fit to the user's existing spectacles.

The manner and accuracy in which the existing spectacles are measured vary from one technician to another. In other words, there isn't any objective or consistent method for accurately and consistently measuring the size of the existing spectacles for the purpose of producing suitable shelter spectacles.

Furthermore, online orderings of spectacles are prevalent in the market. However, such online platforms for purchasing spectacles are not capable of ordering shelter spectacles for existing spectacles worn by users. The major reason is that a uniform and reliable method or platform for measuring the size of existing spectacles is absent.

### SUMMARY OF THE PRESENT INVENTION

The invention is advantageous in that it provides a method and a system for making up spectacles through a predetermined public network, wherein a user is to able to purchase shelter spectacles through the public network.

Another advantage of the invention is to provide a method and a system for making up spectacles through a predetermined public network, wherein the method and system of the present invention is capable of constituting a uniform and reliable method and platform for measuring the size of existing spectacles so as to allow a user to order shelter spectacles without physically going to a store. In other words, a user is able to complete transaction entirely through online ordering.

Another advantage of the invention is to provide a method and a system for making up spectacles through a predetermined public network, wherein a user is able to easily and conveniently order suitable and optimized shelter spectacles through the his or her own computer.

Another advantage of the invention is to provide a method and a system for making up spectacles through a predetermined public network, wherein the measurement of the existing spectacles are standardized under the production system of the present invention so as to generate consistent and reliable measurement results.

Additional advantages and features of the invention will become apparent from the description which follows, and may be realized by means of the instrumentalities and combinations particular point out in the appended claims.

According to the present invention, the foregoing and other objects and advantages are attained by providing a method of purchasing shelter spectacles, comprising the steps of:

(a) gathering dimensional information on an existing spectacles worn by a user;

(b) sending the dimensional information on the existing spectacles to a fulfillment center;

(c) according to the dimensional information sent by the user, producing a shelter spectacles which fit the existing spectacles worn by the user; and

(d) sending the shelter spectacles back to the user.

In accordance with another aspect of the invention, the present invention comprises a shelter spectacles purchasing system, comprising:

a fulfillment center;

a user computer which is arranged to generate a measuring paper having a standardized ruler formed thereon; and

an image capturing device which is electrically connected with the user computer and is arranged to capture an image of a user wearing existing spectacles, wherein the captured image of the existing spectacles is fit into the measuring paper for normalization, wherein the measuring paper with the captured image is sent to the fulfillment center, which is arranged to process the measuring paper and the captured image as normalized by the measuring paper to generate dimensional information of the existing spectacles, wherein the fulfillment center is arranged to assemble a corresponding shelter spectacles according to the dimensional information, wherein the finished shelter spectacles are sent back to the user for wearing

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a block diagram of a method of producing shelter spectacles according to a preferred embodiment of the present invention.

FIG. 2A to FIG. 2D are schematic diagrams of the method of producing shelter spectacles according to the above preferred embodiment of the present invention.

FIG. 3 is a shelter spectacles purchasing system according to the above preferred embodiment of the present invention.

FIG. 4 is a flow chart of method of producing shelter spectacles according to a second preferred embodiment of the present invention.

FIG. 5 illustrates the digital facial image posted on the fulfillment platform according to the above second embodiment of the present invention.

FIG. 6 illustrates the shelter spectacles being produced to match with the existing spectacles according to the first and second embodiment of the present invention.

### DETAILED DESCRIPTION OF THE PREFERRED EMBODIMENT

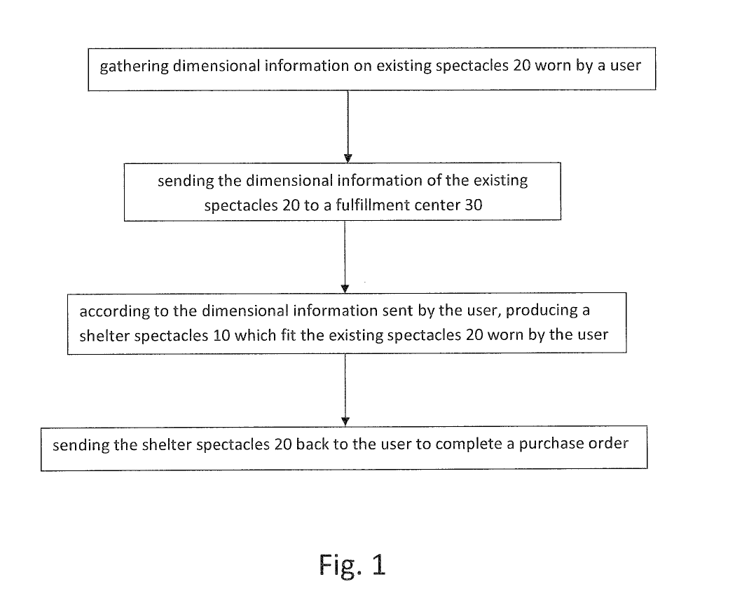

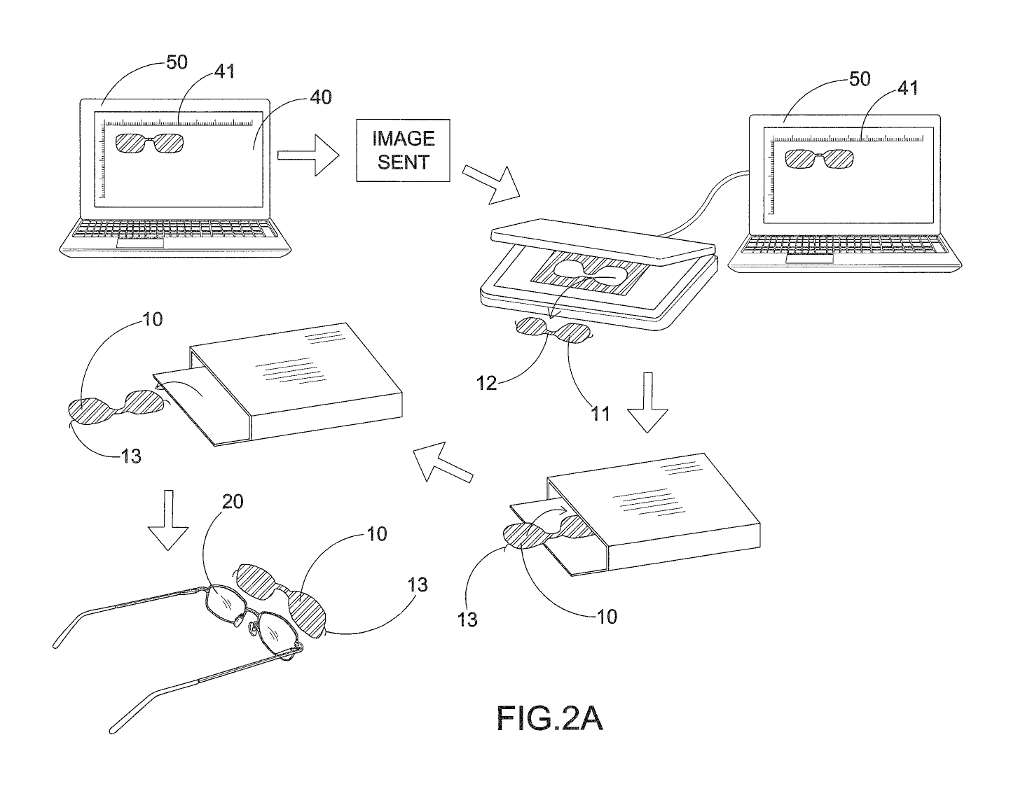

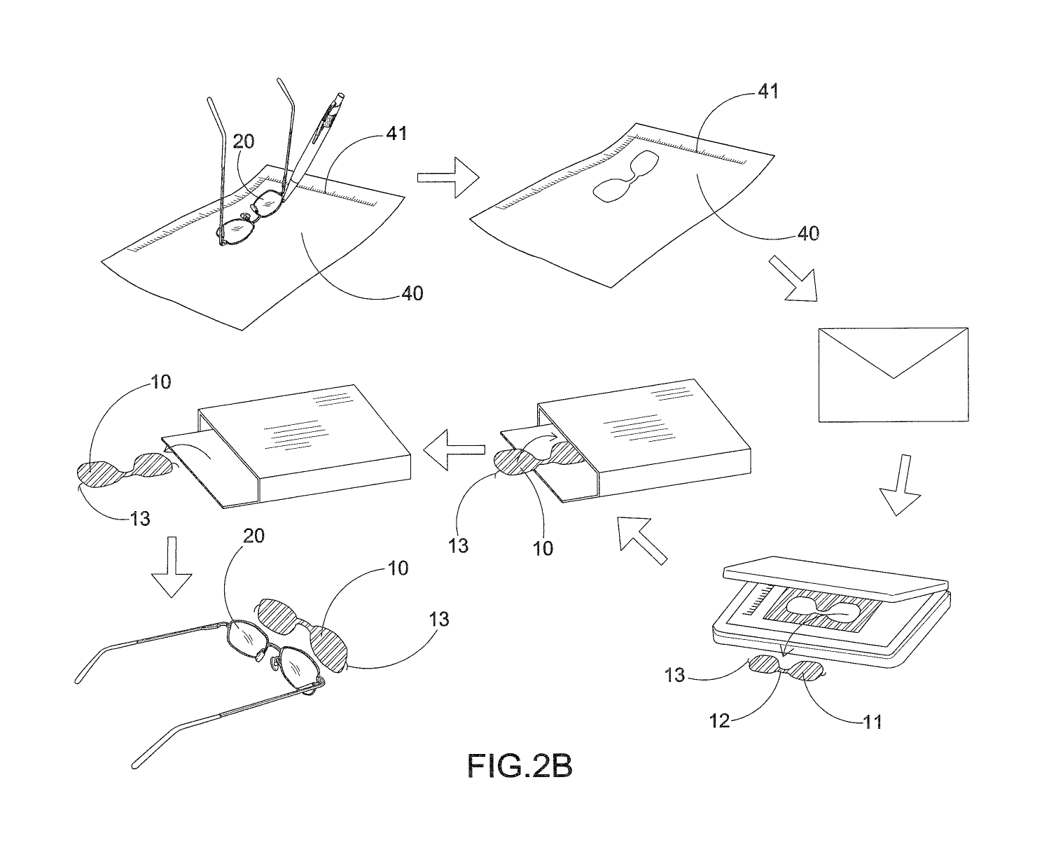

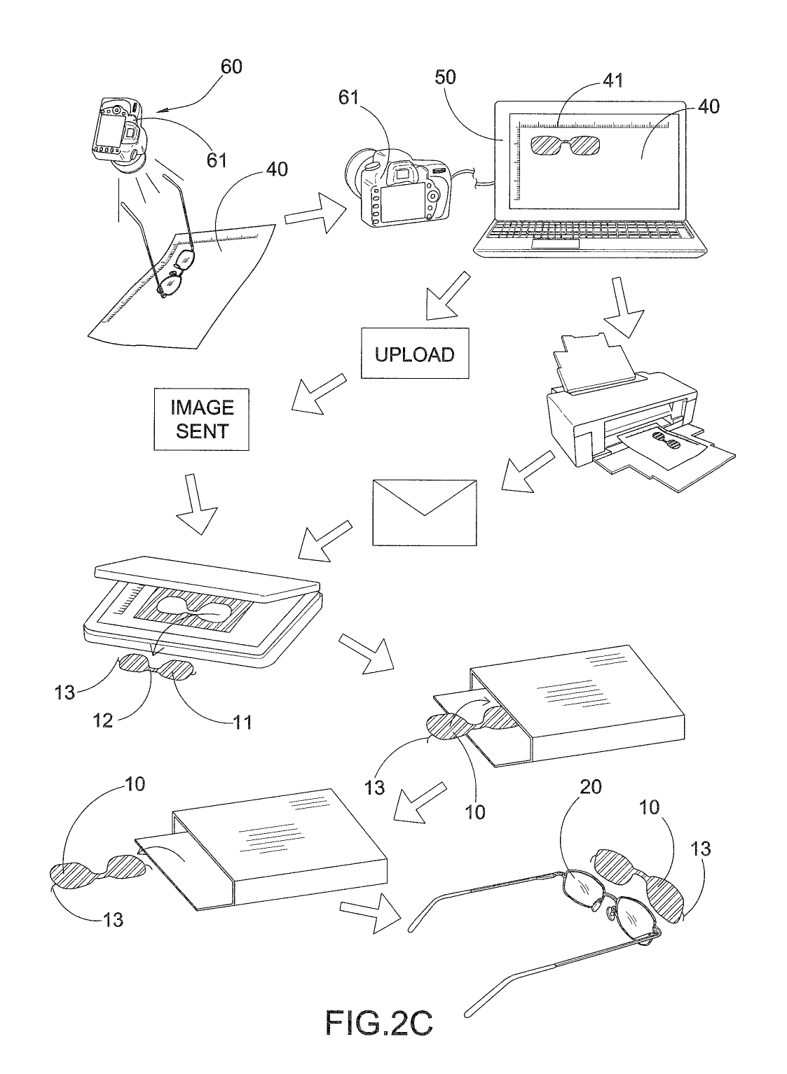

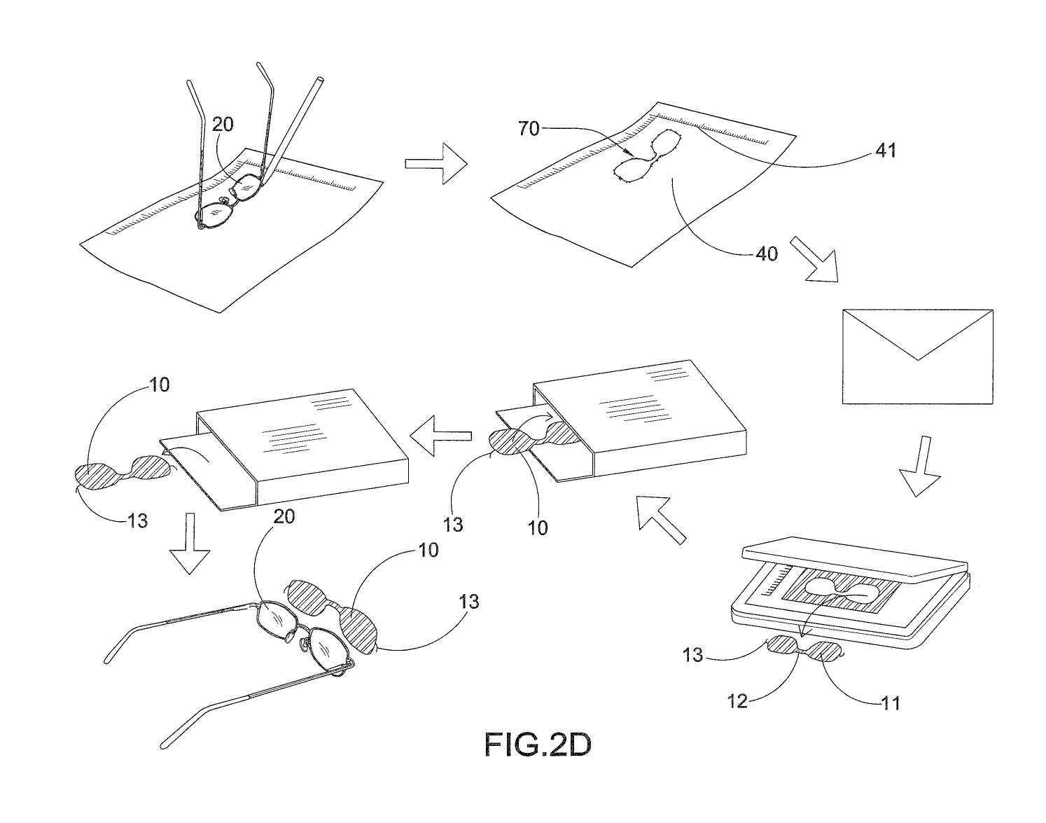

Referring to FIG. 1 and FIG. 2A to FIG. 2D of the drawings, a method of purchasing shelter spectacles 10 according to a preferred embodiment of the present invention is illustrated, in which the method comprises the steps of:

(a) gathering dimensional information on existing spectacles 20 worn by a user (wearer);

(b) sending the dimensional information of the existing spectacles 20 to a fulfillment center 30;

(c) according to the dimensional information sent by the user, producing a shelter spectacles 10 which fit the existing spectacles 20 worn by the user; and

(d) sending the shelter spectacles 20 back to the user to complete a purchase order.

According to the preferred embodiment of the present invention, step (a) comprises the steps of:

(a.1) capturing a digital image of a user wearing the existing spectacles 20;

(a.2) generating a measuring paper 40 having a predetermined number of standardized rulers 41 formed on the measuring paper 40; and

(a.3) printing the digital image of the user wearing the existing spectacles 20 on the measuring paper 40.

It is worth mentioning that by printing the digital image of the existing spectacles 20 on the measuring paper 40, the standardized rulers 41 are arranged to normalize the various dimensions of the existing spectacles (such as height, width, etc.) to the scale used on the measuring paper 40. As such, the manufacturer of the shelter spectacles 10 will know exactly the dimensions and shape of the existing spectacles 20 and is able to produce the corresponding shelter spectacles 10 which fit the existing spectacles 20. In other words, the outline of the existing spectacles 20, i.e. the dimensional information thereof, will be formed on the measuring paper 40.

After the dimensional data of the existing spectacles has been gathered, step (c) comprises the steps of:

(c.1) transforming the dimension of the existing spectacles 20 as appeared on the measuring paper 40 to a real dimension of the shelter spectacles 10;

(c.2) cutting two shelter lenses 11 according to the real dimension of the shelter spectacles 10; and

(c.3) assembling a shelter bridge 12 and a clipping device 13 to the two shelter lenses 11 to form the shelter spectacles 10.

Once the shelter spectacles 10 have been assembled, the finished product will be sent back to the purchaser so that he or she is able to clip the shelter spectacles 10 onto the existing spectacles 20.

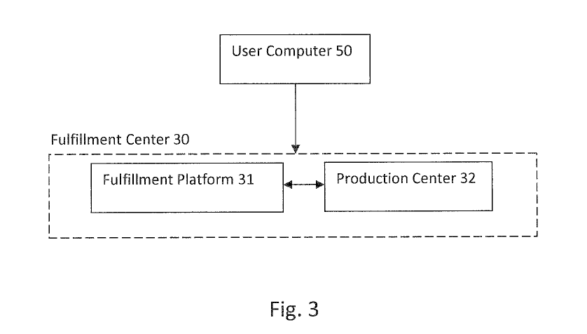

Referring to FIG. 3 of the drawings, a shelter spectacles purchasing system according to the preferred embodiment of the present invention is illustrated, in which the shelter spectacles purchasing system comprises a fulfillment center 30 linked to a user computer 50 which is arranged to generate a measuring paper 40 having a standardized ruler 41 formed thereon, and an image capturing device 60.

The image capturing device 60 is electrically connected with the user computer 50 and is arranged to capture an image of a user wearing existing spectacles 20, wherein the captured image of the existing spectacles 20 is fit into the measuring paper 40 for normalization by the rulers 41, wherein the measuring paper 40 with the captured image is sent to the fulfillment center 30, which is arranged to process the measuring paper 40 and the captured image as normalized by the measuring paper 40 to generate dimensional information of the existing spectacles 20, wherein the fulfillment center 30 is arranged to assemble a corresponding shelter spectacles 10 according to the dimensional information, wherein the finished shelter spectacles 10 are sent back to the user for wearing.

According to the preferred embodiment of the present invention, the fulfillment center 30 comprises a fulfillment platform 31 electrically linked to the user computer 50, and a production center 32 arranged to receive and process the dimensional information from the fulfillment platform 31 and carry out the necessary assembling procedures for producing the shelter spectacles 10.

It is worth mentioning that the measuring paper 40 is essential in allowing the fulfillment center 30 to know the real dimension of the existing spectacles 20. If the user only provides an image of the existing spectacles 20, the fulfillment center 30 will not know the scale of the resulting image and cannot gather the dimensional information of the existing spectacles 20 from the image alone. However, when the existing spectacles are put on the measuring paper having the standardized ruler 41, the fulfillment center 30 is capable of discerning the real dimension of the existing spectacles 20.

The image capturing device 60 comprises a camera 61 electrically connected to the user's computer 50, and is capable of producing images of a predetermined resolution such that the image of the use wearing the existing spectacles 20 can be printed on the measuring paper 40. The measuring paper 40 having the image formed thereon is then sent to the fulfillment center 30.

In order to allow the user to select a wide range of shelter spectacles 10, the fulfillment center 30 further comprises a spectacles database 33 electrically linked to the fulfillment platform 31, wherein the spectacles database 33 is arranged to store images of a plurality of shelter spectacles 10 for user's selection. Thus, the images of the shelter spectacles 10 are arranged to be accessed by the user's computer 50 for selection. The user, upon confirmation of a selection, may send the measuring paper 40 to the fulfillment center 30 for production of selected shelter spectacles 10.

It is also important to point out that there exists a number of alternatives as to the manner in which dimensional information of the existing spectacles 20 can be gathered. Thus, there exists a number of alternatives for the above-mentioned step (a). These alternatives are described as follows.

In its first alternative, step (a) comprises the steps of:

(a.1′) printing a measuring paper 40 having a predetermined number of standardized rulers 41 formed on the measuring paper 40; and

(a.2′) putting the existing spectacles 20 on the measuring paper 40; and

(a.3′) producing a drawing on the measuring paper 40 following a contour of the existing spectacles 20.

Thus, instead of taking a photo of the user wearing the existing spectacles 20, the user may actually make a drawing of his or her existing spectacles 20 and send the drawing to the fulfillment center 30. Since the drawing is produced on the measuring paper 40, the dimensional information can be gathered by relying on the dimension as shown in the drawing.

In these alternative steps, step (b) comprises a step of mailing the measuring paper 40 having the drawing produced thereon to the fulfillment center 30 so as to allow the fulfillment center 30 to produce the correspond shelter spectacles 10.

As a second alternative, step (a) comprises the steps of:

(a.1″) printing a measuring paper 40 having a predetermined number of standardized rulers 41 formed on the measuring paper 40; and

(a.2″) putting the existing spectacles 20 on the measuring paper 40;

(a.3″) producing a drawing on the measuring paper 40 following a contour of the existing spectacles 20; and

(a.4″) capturing an image of the measuring paper 40 with the drawing of the existing spectacles 20 produced on the measuring paper 40.

When the image of the measuring paper 40 is captured, the image may be electronically sent to the fulfillment center 30. Alternatively, the image may be printed on a separate paper and physically mailed to the fulfillment center 30.

As a third alternative, step (a) comprises the steps of:

(a.1a) printing a measuring paper 40 having a predetermined number of standardized rulers 41 formed on the measuring paper 40; and

(a.2a) putting the existing spectacles 20 on the measuring paper 40; and

(a.3a) producing a plurality of puncture points 70 on the measuring paper 40 at predetermined peripheral points of the existing spectacles 20 so as to allow the fulfillment center 30 to measure the dimensional information of the existing spectacles 20 by measuring the corresponding distance of the puncture points.

In step (a.3a) mentioned above, the puncture points 70 produced on the measuring paper 40 are positioned in accordance with a peripheral edge of the existing spectacles 20. In other words, the puncture points 70 illustrates the contour of the existing spectacles 20 and allow the fulfillment center 30 to gather the essential dimensional information of the existing spectacles 20.

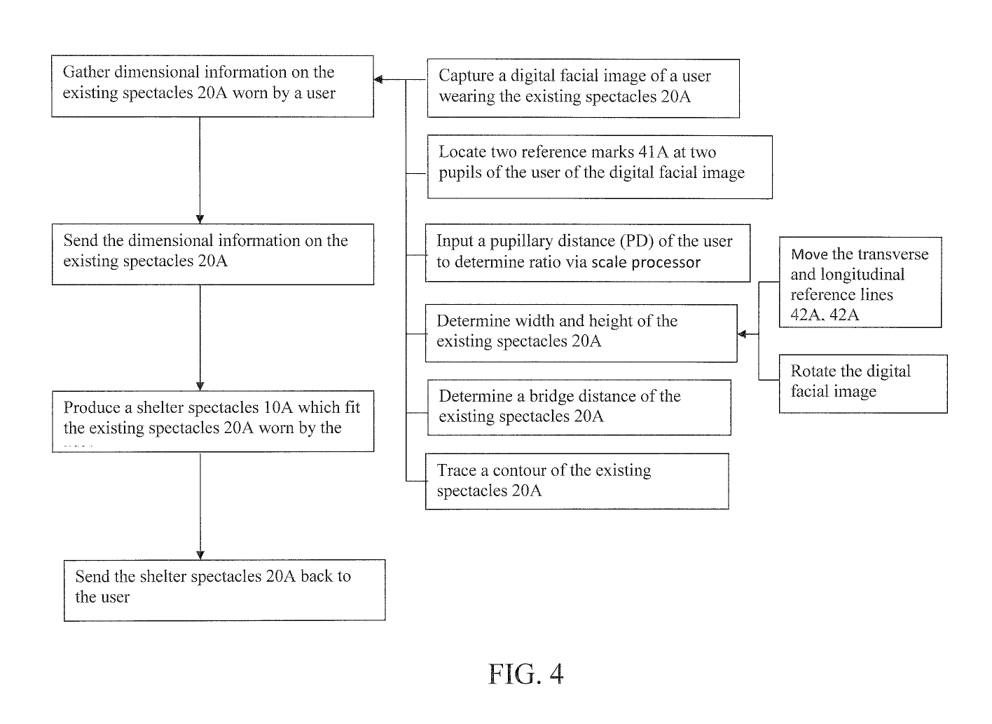

As shown in FIG. 4, a method of making a shelter spectacle 10A through a public network according to second preferred embodiment illustrates an alternative mode of the present invention, wherein the method comprises the following steps.

(1) Gather dimensional information on existing spectacles 20A worn by a user.

(2) Send the dimensional information of the existing spectacles 20A to a fulfillment center 30A through the public network.

(3) According to the dimensional information sent by the user, produce a pair of shelter spectacles 10A which fit the existing spectacles 20A worn by the user.

(4) Send the shelter spectacles 20A back to the user to complete a purchase order.

In the step (1), the dimensional information of the existing spectacles 20A is obtained by the following steps.

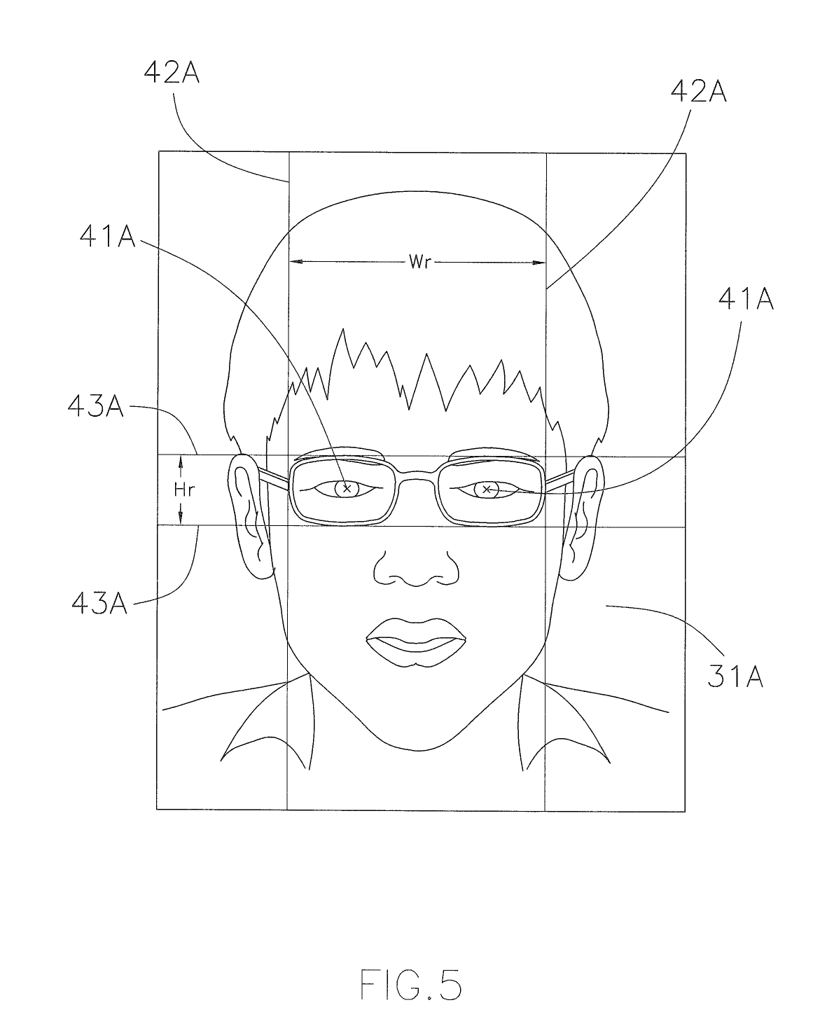

(1.1) Capture a digital facial image of a user (wearer) wearing the existing spectacles 20A. In particular, a full frontal photo as the facial image is captured to show the user wearing the existing spectacles 20A. The digital facial image can be posted on the user computer 50A and/or the fulfillment platform 31A of the fulfillment center 30A. Preferably, the digital facial image of the user is directly posted on the fulfillment platform 31A of the fulfillment center 30A as a posting platform as shown in FIG. 5. Accordingly, when the full frontal photo of the user is posted on the user computer 50A and/or the fulfillment platform 31A of the fulfillment center 30A, the digital facial image will show two pupils of the user and the frontal photo of the existing spectacles 20A on the full frontal photo of the user. It is worth mentioning that the fulfillment platform 31A of the fulfillment center 30A communicatively linked to the user computer 50 via communication network such as Internet. In other words, the user is able to directly link to the fulfillment platform 31A via the user computer 50A in order to post the digital facial image on the fulfillment platform 31A.

(1.2) Locate two reference marks 41A at the pupils of the user of the digital facial image, as shown in FIG. 5. In the step (1.2), when the two reference marks 41A are located at the pupils of the wearer of the digital facial images, the scale processor of the fulfillment platform 31A will determine the distance between the two reference marks 41A to represent the distance of the pupils of the user.

(1.3) Input the pupillary distance (PD) of the user. Accordingly, the pupillary distance rang of a user is between 57 and 65 mm. The pupillary distance of the user is able to obtain from the eye doctor or other eye care professionals. During the eye examination, the pupillary distance will be measured and shown in the glasses prescription. The pupillary distance can also be measured by the user himself or herself by bringing a ruler directly below the center of the one eye's pupil and looking straight into the mirror. Once the other eye's pupil is located and marked on the ruler, the pupillary distance can be determined. The user can repeat the above ruler measuring process to ensure the accurate measurement of the pupillary distance. According to the preferred embodiment, once a real pupillary distance of the user is input, a face scale of the face image will be determined by comparing the real pupillary distance with the distance between the two reference marks 41A.

(1.4) Determine the width and height of the existing spectacles 20A. The width and height of the existing spectacles 20A is determined by a reference width Wr and reference height Hr of the existing spectacles 20A at the digital facial image. Accordingly, the user is able to pin-point the outline of the existing spectacles 20A on the digital facial image to indicate the reference width Wr and reference height Hr of the existing spectacles 20A thereon. Preferably, two transverse reference lines 42A are selectively moved by the user until the transverse reference lines 42A are located at the outer sides of the existing spectacles 20A on the digital facial image to indicate the reference width Wr of the existing spectacles 20A. Accordingly, the reference width Wr of the existing spectacles 20A does not include the temples thereof. Two longitudinal reference lines 43A are selectively moved by the user until the longitudinal reference lines 43A are located at the top and bottom sides of the existing spectacles 20A on the digital facial image to indicate the reference height Hr of the existing spectacles 20A. The fulfillment center 30 will read the reference width Wr and the reference height Hr corresponding to the distance between the two transverse reference marks 42A and the distance between the two longitudinal reference lines 43A via the scale processor. Accordingly, the two transverse reference lines 42A are perpendicular to the longitudinal reference lines 43A. Therefore, having the ratio of the pupillary distance (PD) input by the user and the distance between the two reference marks 41A, the fulfillment center 30A will know the real dimension of the existing spectacles 20A, i.e. the width and height of the existing spectacles 20A via the scale processor.

For calibrating the outline of the existing spectacles 20A with respect to the transverse reference lines 42A and the longitudinal reference lines 43A, the digital facial image can be rotated to match with the transverse reference lines 42A and the longitudinal reference lines 43A. In other words, the transverse reference lines 42A are two vertical reference lines and the longitudinal reference lines 43A are two horizontal reference lines, wherein the digital facial image is rotated until the transverse reference lines 42A and the longitudinal reference lines 43A form a rectangular box configuration to match with the outline of the existing spectacles 20A on the digital facial image.

Alternatively, the transverse reference lines 42A are two inclined reference lines extending in parallel to match with the offset of the existing spectacles 20A on the digital facial image. It is worth mentioning that the two transverse reference lines 42A must be perpendicular to the longitudinal reference lines 43A.

Once the outline of the existing spectacles 20A on the digital facial image is determined, the production center 32A will receive and process the dimensional information from the fulfillment platform 31A and carry out the necessary assembling procedures for producing the shelter spectacles 10A.

It is worth mentioning that the fulfillment platform 31A will automatically determine the bridge distance of the existing spectacles 20A in response to the outline of the existing spectacles 20A on the digital facial image.

Having the ratio of the pupillary distance (PD) input by the user and the distance between the two reference marks 41A, the fulfillment center 30A will know the real dimension of the existing spectacles 20A, especially with the outline of the existing spectacles 20A on the digital facial image by the transverse reference lines 42A and the longitudinal reference lines 43A. In other words, the fulfillment center 30A is capable of discerning the real dimension of the existing spectacles 20A. The contour of the existing spectacles 20A will be automatically traced and determined by the production center 32A in order to produce the shelter frame 10A which fit the existing spectacles 20A worn by the user. It is worth mentioning that once the outline of the existing spectacles 20A on the digital facial image is determined by the transverse reference lines 42A and the longitudinal reference lines 43A, the contour of the existing spectacles 20A can be tracked by computer software or by a skilled technician of the fulfillment center 30A. In addition, the contour of the existing spectacles 20A is formed in proportion to the real existing spectacles 20A. It is worth mentioning that once the contour of the existing spectacles 20A is formed, every single point at the peripheral edge of the existing spectacles 20A at the digital facial image will be proportionally found with respect to the existing spectacles 20A so as to determine the bridge distance of the existing spectacles 20A.

In order to allow the user to select a wide range of shelter spectacles 10A, the fulfillment center 30A further comprises a spectacles database 33A electrically linked to the fulfillment platform 31A, wherein the spectacles database 33A is arranged to store images of a plurality of shelter spectacles 10A for user's selection. Thus, the images of the shelter spectacles 10A are arranged to be accessed by the user's computer 50A for selection.

After the dimensional data of the existing spectacles has been gathered, the step (3) comprises the following steps.

(3.1) Transform the dimension of the existing spectacles 20A to a real dimension of the shelter spectacles 10A.

(3.2) Cut two shelter lenses 11A according to the real dimension of the shelter spectacles 10A. The size of each of the shelter lens 11A matches with the size of the corresponding lens of the existing spectacles 20A.

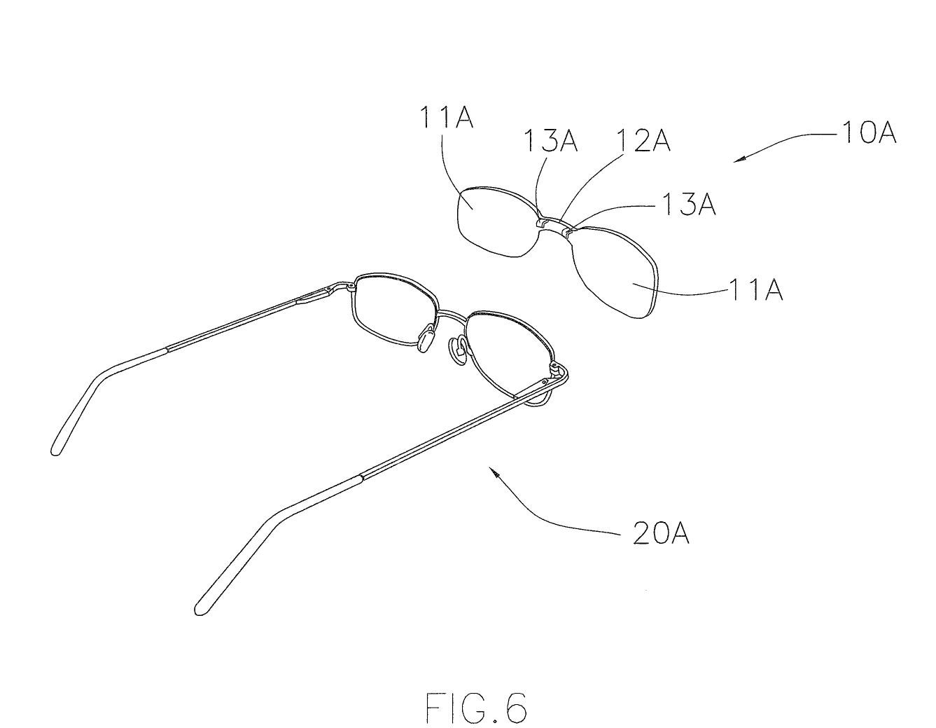

(3.3) Assemble a shelter bridge 12A and a clipping device 13A to the two shelter lenses 11A to form the shelter spectacles 10A. As it is mentioned above, the bridge distance of the existing spectacles 20A is determined, the length of the shelter bridge 12A can be formed to match with the corresponding bridge of the existing spectacles 20A. The clipping device 13A is also formed at the inner sides of the shelter lenses 11A to detachably couple with the inner sides of the lenses of the existing spectacles 20A, as shown in FIG. 6. It is appreciated that the clipping device 13 can be formed at two outer sides of the shelter lenses 11A according to the first embodiment or can be formed at two inner sides of the shelter lenses 11A according to the second embodiment, wherein the locations of the clipping device can be interchangeable in the embodiments.

Once the shelter spectacles 10A have been assembled, the finished product will be sent back to the purchaser so that he or she is able to clip the shelter spectacles 10A onto the existing spectacles 20A.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method of making a shelter spectacle for existing spectacles through a public network, comprising the steps of:
(a) gathering dimensional information on said existing spectacles worn by a user;
(b) sending said dimensional information on said existing spectacles to a fulfillment center through the public network;
(c) according to said dimensional information sent by said user, producing a shelter spectacles which fit said existing spectacles worn by said user; and
(d) sending said shelter spectacles back to said user.

2. The method, as recited in claim 1, wherein the step (a) further comprises the steps of:
(a.1) capturing a digital facial image of a user wearing said existing spectacles, wherein said digital facial image of said user is a full frontal photo of said user;
(a.2) selectively locating two reference marks at two pupils of said user of said digital facial image; and
(a.3) inputting a pupillary distance (PD) of said user in order to determine a ratio of said pupillary distance of said user and a distance between said reference marks on said digital facial image.

3. The method, as recited in claim 2, wherein the step (a) further comprises a step of (a.4) determining width and height of said existing spectacles by a reference width and a reference height of said existing spectacles at said digital facial image.

4. The method, as recited in claim 3, wherein the (a.4) further comprising the steps of:
(a.4.1) selectively moving two transverse reference lines on said digital facial image until said transverse reference lines are located at two outer sides of said existing spectacles on said digital facial image to indicate said reference width of said existing spectacles so as to determine said width of said existing spectacles; and
(a.4.2) selectively moving two longitudinal reference lines on said digital facial image until said longitudinal reference lines are located at top and bottom sides of said existing spectacles on said digital facial image to indicate said reference height of said existing spectacles so as to determine said height of said existing spectacles.

5. The method, as recited in claim 4, wherein said two transverse reference lines are perpendicular to said longitudinal reference lines.

6. The method, as recited in claim 5, wherein the (a.4) further comprising a step of rotating said digital facial image to match with said transverse reference lines and said longitudinal reference lines, wherein said transverse reference lines are two vertical reference lines and said longitudinal reference lines are two horizontal reference lines.

7. The method, as recited in claim 2, wherein the step (a) further comprises a step of automatically determining a bridge distance of said existing spectacles in response to said existing spectacles on said digital facial image.

8. The method, as recited in claim 6, wherein the step (a) further comprises a step of automatically determining a bridge distance of said existing spectacles in response to said existing spectacles on said digital facial image.

9. The method, as recited in claim 2, wherein the step (a) further comprises a step of automatically tracing a contour of said existing spectacles in order to form said shelter spectacles.

10. The method, as recited in claim 8, wherein the step (a) further comprises a step of automatically tracing a contour of said existing spectacles in order to form said shelter spectacles.

11. The method, as recited in claim 10, wherein said step (c) comprises the steps of:
(c.1) transforming said dimension of said existing spectacles on said digital facial image to a real dimension of said shelter spectacles;
(c.2) cutting two shelter lenses according to said real dimension of said shelter spectacles; and
(c.3) assembling a shelter bridge and a clipping device to said two shelter lenses to form said shelter spectacles.

12. The method, as recited in claim 1, wherein said step (a) comprises the steps of:
(a.1) capturing a digital image of a user wearing said existing spectacles;
(a.2) generating a measuring paper having a predetermined number of standardized rulers formed on said measuring paper, wherein said standardized rulers are arranged to normalize various dimensions of said existing spectacles to a scale used on said measuring paper; and
(a.3) printing said digital image of said user wearing said existing spectacles on said measuring paper.

13. The method, as recited in claim 12, wherein said step (c) comprises the steps of:
(c.1) transforming said dimension of said existing spectacles as appeared on said measuring paper to a real dimension of said shelter spectacles;
(c.2) cutting two shelter lenses according to said real dimension of said shelter spectacles; and
(c.3) assembling a shelter bridge and a clipping device to said two shelter lenses to form said shelter spectacles.

14. A shelter spectacles production system, comprising:
a fulfillment platform adapted for gathering dimensional information on an existing spectacles worn by a user and receiving said dimensional information on said existing spectacles through the public network; and
a production center linked to said fulfillment platform for producing shelter spectacles which fit said existing spectacles worn by said user according to said dimensional information sent by said user.

15. The system, as recited in claim 14, wherein said fulfillment platform comprises a posting platform for posting a digital facial image of a user, which is a full frontal photo, wearing said existing spectacles, two reference marks selectively locating at two pupils of said user of said digital facial image, and a scale processor determining a ratio of a pupillary distance of said user and a distance between said reference marks on said digital facial image when said pupillary distance (PD) of said user is input to said fulfillment platform.

16. The system, as recited in claim 14, wherein said fulfillment platform further comprises two transverse reference lines and two longitudinal reference lines, wherein said two transverse reference lines are selectively moved on said digital facial image until said transverse reference lines are located at two outer sides of said existing spectacles on said digital facial image to indicate a reference width of said existing spectacles for determining a width of said existing spectacles, wherein said two longitudinal reference lines are selectively moved on said digital facial image until said longitudinal reference lines are located at top and bottom sides of said existing spectacles on said digital facial image to indicate said reference height of said existing spectacles for determining said height of said existing spectacles.

17. The system, as recited in claim 16, wherein said two transverse reference lines are perpendicular to said longitudinal reference lines.

18. The system, as recited in claim 17, wherein said fulfillment platform further comprises means for automatically determining a bridge distance of said existing spectacles in response to said existing spectacles on said digital facial image, and means for automatically tracing a contour of said existing spectacles in order to form said shelter spectacles.

19. The system, as recited in claim 15, further comprising a spectacles database electrically linked to said fulfillment platform, wherein said spectacles database is arranged to store images of a plurality of shelter spectacles for user's selection, wherein said images of said shelter spectacles are arranged to be accessed by said user's computer for selection.

20. The system, as recited in claim 18, further comprising a spectacles database electrically linked to said fulfillment platform, wherein said spectacles database is arranged to store images of a plurality of shelter spectacles for user's selection, wherein said images of said shelter spectacles are arranged to be accessed by said user's computer for selection.
